(19) United States
(12) Patent Application Publication
(10) Pub. No.: US 2015/0305965 A1
(43) Pub. Date: Oct. 29, 2015

(54) READING THERAPY

(71) Applicant: King-Devick Test, LLC, Oakbrook Terrace, IL (US)

(72) Inventor: Steven Devick, Downers Grove, IL (US)

(73) Assignee: King-Devick Test, LLC, Oakbrook Terrace, IL (US)

(21) Appl. No.: 14/593,122

(22) Filed: Jan. 9, 2015

Related U.S. Application Data
(60) Provisional application No. 61/985,021, filed on Apr. 28, 2014.

(51) Int. Cl. A61H 5/00; G09B 17/00; A61B 3/02

(52) U.S. Cl. CPC A61H 5/00; A61B 3/02; G09B 17/003

(57) ABSTRACT

Systems and methods for improving saccadic eye movement are disclosed.

### CROSS-REFERENCE TO RELATED APPLICATIONS

This application claims the benefit of and priority to U.S. provisional patent application Ser. No. 61/985,021, titled “READING THERAPY”, filed Apr. 28, 2014, which is hereby incorporated herein by reference in its entirety.

### BACKGROUND

The King-Devick Test (the “K-D Test” or “Test”) is widely recognized as a tool to evaluate saccadic eye movements. The Test is widely used by reading teachers in schools, medical professionals, or parents at home to determine whether a student's poor reading performance is related to deficiencies in their ability to move their eyes efficiently. Children that score below the expected norm are, almost always, not efficient readers. The correlation between slower and/or less accurate saccadic eye movement, measured by the K-D Test, and lower reading fluency, has been well-established.

The inventor realized that, in addition to testing saccadic eye movement, activities like the K-D Test (but not limited to the K-D Test) could be used to practice saccadic eye movement, and could be designed to encourage fast and accurate saccadic eye movement. Moreover, the inventor realized that, if a student's reading fluency was being hindered by inefficient saccadic eye movement, that student's saccadic eye movement, and reading fluency, could be improved by practicing a saccadic-eye-movement-intensive activity, like taking the K-D Test.

Similarly, the inventor realized that saccadic eye movement in those suffering from brain injury could be improved by practice. Patient who have suffered a traumatic brain injury can often exhibit problems in reading. While some such reading problems can be cognitively based, for example those related to aphasia, agraphia, etc., in other cases difficulty reading can be based on a physiologically-based inability to perform fast and accurate saccadic eye movement. By practicing saccadic eye movement, such as the saccadic eye movement required by the K-D Test, patients recovering from brain injuries can improve their saccadic eye movement and concomitantly improve their reading.

### SUMMARY

Systems and methods for improving saccadic eye movement are disclosed.

### BRIEF DESCRIPTION OF THE DRAWINGS

FIG. 1 depicts a demonstration page of a K-D Test booklet.

### DETAILED DESCRIPTION

A method can include using a computer, the computer including a visual output device, the computer being configured to visually display numbers in predetermined spatial and temporal arrangements on the visual output device. The method can include (a) visually presenting the visual output device to a trainee; (b) instructing the trainee to read aloud the numbers in the predetermined spatial and temporal arrangement in a specified order without moving the trainee's head; (c) visually displaying to the trainee a predetermined spatial and temporal arrangement of numbers on the visual output device; (d) reading aloud the numbers in the visually displayed predetermined spatial and temporal arrangement, wherein reading is to be carried out by the trainee without moving the trainee's head, thereby causing the trainee to engage in saccadic eye movement while reading the numbers; and (e) repeating steps (a), (b), (c) and (d) on a predetermined schedule. The trainee's act of reading aloud the numbers in the spatial or spatial and temporal arrangement without moving his/her head forces the trainee to engage in saccadic eye movement, just like in an administration of the K-D Test.

The trainee can be any person desiring to improve speed and/or accuracy of saccadic eye movement. For example, the trainee could be someone diagnosed with traumatic brain injury. Or the trainee could be someone diagnosed with dyslexia. Or the trainee could be a person found to have slow or inaccurate saccadic eye movement for any other reason.

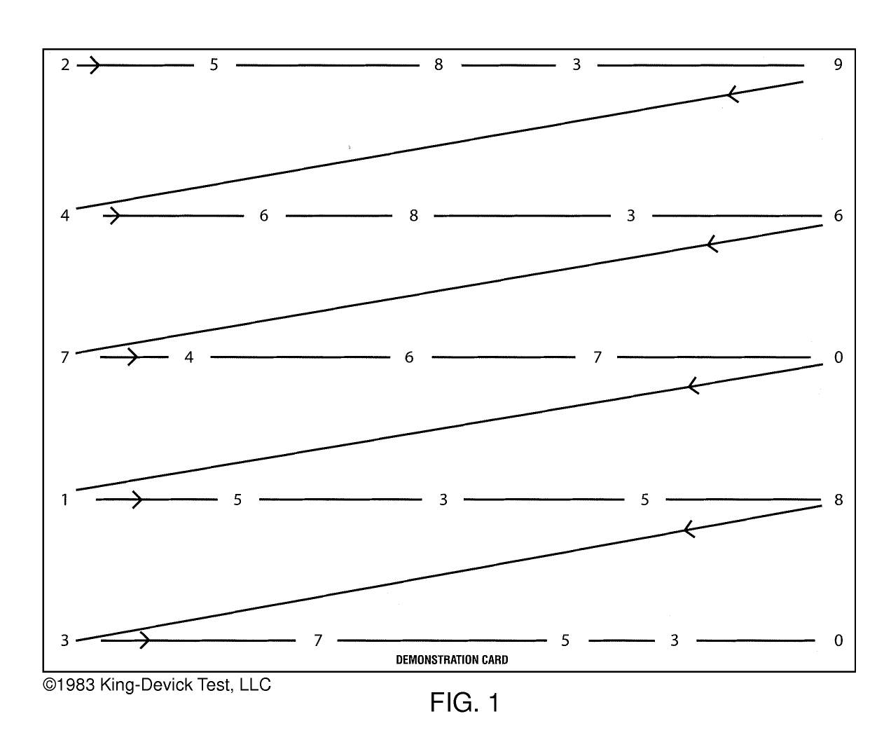

In some such methods, the predetermined spatial and temporal arrangement of numbers includes numbers spaced apart along a sequence of vertically spaced apart horizontal axes. For example, FIG. 1 shows a demonstration page from a K-D test booklet. Numbers are arranged along five vertically spaced apart horizontal axes. The horizontal spacing of the numbers varies between axes. On the second line the numbers are relatively evenly spaced, while on the last line the numbers are more bunched toward the right end of the row. The horizontal spacing on each row can be different, and may or may not be related to the horizontal spacing on any other row. Because this is a demonstration page, the numbers are connected by lines and arrows that visually explain the order in which the numbers should be read, left to right along each horizontal axis, and each horizontal axis from top to bottom. A test card, unlike a demonstration card, might or might not include lines and/or arrows between the numbers. Arrangements that are relatively easy to read can include, for example, any combination of (a) relatively even horizontal spacing between the numbers along a horizontal axis, (b) relatively even vertical spacing of horizontal axes, (c) numbers at the left and right ends of each horizontal axis, and/or (d) a large font. Arrangements that are more difficult to read can incorporate, for example, any combination of (a) more uneven spacing, (b) no lines between numbers, (b) smaller numbers, and/or (d) a lack of numbers at the right- and left-most points on some or all horizontal axes.

The arrangement need not be an arrangement of numbers used in the K-D Test. For example, the method can make use of a variety of different cards. The numbers can be arranged according to an algorithm, or can be arranged randomly, partially randomly, or pseudo-randomly. The cards can be of varying difficulty, so that a user who is able to complete easier cards quickly and/or with few or no mistakes can move on to practice more difficult cards.

Similarly, if implemented on a computer, the user can graduate from easier arrangements, to more difficult ones. If implemented on a computer, the various arrangements can be saved as a static collection of arrangements, or can be generated as needed. If generated, they can be generated according to a predetermined algorithm, randomly, pseudo-randomly, or partially randomly. For example, certain aspects of the arrangement may be randomized, like which number is used in a given position, while other aspects might be static, like the spacing of the numbers. Any spatial arrangement that can be displayed on a card can also be displayed on an electronic visual output, like the visual display of a computer or similar device.

On a device capable of a transient or partially transient display of the arrangement of numbers, like a computer with a display screen, but unlike a printed test booklet, the arrangement of numbers can be an arrangement in both space and time. For example: the numbers could appear one at a time; each number could disappear before the next appears; the numbers could all be displayed simultaneously; only a few numbers could be displayed at a time; the numbers could all appear simultaneously and then disappear one-by-one; or any other spatial-temporal arrangement of numbers.

The numbers can be of any length, for example single-digit numbers as shown in FIG. 1, or multi-digit numbers. Instead of numbers, the methods disclosed herein could make use of letters, symbols, pictograms, pictures, or anything else that can be inspected with saccadic eye movement. For clarity, the remainder of this description focuses solely on embodiments using single-digit numbers even though other embodiments are covered by this disclosure.

In administering a K-D Test to test subject, a test administrator (or an activated computer) keeps track of the time the test subject takes to read the predetermined arrangement, and the number of errors the test subject makes. Similarly, time and error rate can be measured and recorded for a trainee practicing saccade. In some embodiments, timing and/or error rate can be used to tailor the arrangement of numbers to the ability of the trainee. A trainee that is reading an arrangement quickly and/or making few or no errors, is presumably not being challenged by the current arrangement. The arrangement displayed the next time the method is repeated can be selected to increase the difficulty. Regardless of whether the difficulty is changed, the particular arrangement displayed will typically be non-identical on subsequent iterations. In some cases, no two displayed arrangements will be identical. In some cases, some level of repetition of displayed arrangements is acceptable.

Time taken to read the predetermined arrangement can be measured by the computer if one is being used, or can be recorded by a human trainer. If recorded by a human trainer, the measured time can be recorded manually on a computer if used, or by hand, for example if no computer is being used. The same is true for error rates; a computer can automatically detect and count errors, or a human trainer can count errors and input the count to a computer, or a human trainer can count errors and manually record them, for example if no computer is being used. The measured time or error rate can be used to dynamically adjust the difficulty of the arrangement. For example, if the trainee is able to read an arrangement in a relatively short amount of time, a subsequent arrangement may be chosen so as to reduce the amount of time available to the trainee. If the numbers are displayed one at a time, each for a limited time, the time for which each number is displayed may be shortened. Or the spatial characteristics may be made more difficult to read. The same can be true of the trainee has a low or zero error rate. The predetermined arrangement itself can be adjusted to make it more difficult, or a more difficult predetermined arrangement can be selected.

When implemented with a computer, voice recognition tools can be incorporated into the system so that the computer automatically measures the time the trainee takes to read the arrangement and automatically counts the number of errors the trainee makes while reading the visual display.

In embodiments where each number is displayed only for a predetermined time, the predetermined time can be, for example, less than 3 seconds, less than 2 seconds, from 0.25 to 1.6 seconds, about 1 second, or about 0.5 seconds. In some embodiments, only one number is displayed at a time. In some embodiments, all numbers are simultaneously displayed. In some embodiments, all numbers appear simultaneously, but disappear one by one in the order in which they are intended to be read.

Training can be carried out on a predetermined schedule. The schedule can include reading a predetermined number of predetermined arrangements, or can be based on an amount of time spent reading predetermined arrangements. For example, the schedule could include reading 1, 2, 5, 10, 20, 30, 50, or 100 predetermined arrangements on a given day. The schedule could include training for 1, 2, 5, 10, 20, 30 or 60 minutes on a given day. The schedule could include carrying out a given day's training all at once, or broken into different segments. The schedule could include training 1, 2, 3, 4, 5, 6 or 7 days a week. The schedule could include training for a set number of weeks, or the schedule could include training until desired benchmarks are met, no matter how long that takes. The desired benchmarks could include, for example, a certain time and/or error rate on the K-D Test or similar test of saccadic eye movement, or a certain level of reading fluency.

In general, a method of reading fluency can include identifying a trainee in need of improved reading fluency; the trainee undertaking a predetermined series of saccadic eye movements; the trainee repeating the predetermined series of saccadic eye movements on a predetermined schedule, thereby improving the trainee's reading fluency.

In cases where the training is administered on a computer, the computer can be connected to a memory, either locally, or remotely on a server connected through a network. The memory can record, for example, a signal representative of the fact that the trainee has attempted to carry out the method, the fact that the trainee spent a certain amount of time carrying out the method, the time the trainee took to read aloud the predetermined arrangement or arrangements, the trainee's error rate in such reading, etc. Such data can be stored in the memory and tracked to show progress and/or compliance (or lack thereof) by the trainee.

### Example

In a prospective, single-blinded, randomized, crossover trial, a cohort of elementary students received standardized reading fluency testing pre- and post-treatment. Treatment consisted of in-school training 20 minutes per day, 3 days per week for 6 weeks.

Study Participants

Subjects in this pilot study were students from a private urban elementary school. All students enrolled in Kindergarten through 3rd grade completed a school vision screening. Information about the study and an invitation to participate was provided to the parent or guardian of students' whose binocular habitual near point acuity was 20/20. Recruited subjects were randomized into two groups with an approximately 3:1 ratio: treatment (n=56) and placebo (n=20). Written informed consent was obtained from all participants' parent or guardian and child assent was obtained from all participants.

Reading Fluency Assessment: The Wechsler Individual Achievement Test 3rd Edition (WIAT)

For the standardized reading fluency assessment, the Reading Fluency Subtest of the Wechsler Individual Achievement Test 3rd Edition (WIAT) was utilized in this study for students in grades 1st through 3rd. In the Reading Fluency subtest of the WIAT, participants are timed while reading two grade-level specific passages aloud. It is designed to assess the speed and accuracy of the participant's reading ability and identify academic weaknesses. The total number of words read, number of errors made, and total time are recorded. The WIAT reading score is based on an average word read correctly per minute (WCPM) objective measurement and a percentile ranking is determined.

The King-Devick Test

The K-D test was utilized in this study for kindergarten students due to the unavailability of a reading fluency assessment for this age group. The K-D test is based on a measurement of saccadic and number naming speed. The test involves reading aloud a series of randomized single numbers from left to right. The K-D test includes one demonstration card and 3 test cards that increase in difficulty. The score is based on the cumulative time taken to read each card. The number of errors made in reading the test cards is also recorded. In this study, standardized instructions were used and participants were asked to read the numbers from left to right as quickly as they could without making any errors. A K-D Composite score was calculated by adding the time score and error score together. A lower K-D Composite score indicated higher efficiency and accuracy.

The King-Devick Remediation Software

The King-Devick remediation software is similar in format to the K-D test. It is available on a computer or a mobile iPad. Randomized numerical stimuli are presented at variable speeds from left to right. Participants were asked to the read aloud the numbers from left to right as quickly as they could without making errors. The computer based K-D remediation software was utilized in this study.

Treatment Protocol

In this prospective, single-blinded, randomized, crossover study clinical trial, subjects were assessed pre- and post-treatment to determine reading fluency performance. Subjects were tested using the WIAT and K-D tests. A previous study has shown that the K-D test can be completed by kindergarteners and that the performance on the K-D test is related to academic performance in kindergarteners.26 Due to the unavailability of a reading fluency measurement for the kindergarten age and school level, only the K-D test was used for pre- and post-treatment assessments in kindergarteners. Pre-treatment testing was conducted in the subjects' respective school classrooms by trained test administrators masked to the sequence of intervention. The treatment consisted of 6 weeks of saccadic training using K-D remediation software conducted by independent teachers, teaching aides and graduate students all trained to use the K-D remediation software with students. All treatment sessions were conducted in the school computer room. The treatment protocol consisted of 20 minute individual training sessions, 3 days each week for 6 weeks for a total of 6 hours of training. In the treatment group, numerical stimuli were presented in a left to right orientation on the computer screen and the speed of presentation was varied from 1600 milliseconds to 250 milliseconds. Subjects were instructed to read the presented number aloud as quickly as they could without making errors. As subjects improved, the speed of rapid number presentation was regularly increased.

The control group was presented with individual numbers positioned in the center of the computer screen, which did not change positions. The speed of presentation was varied from 1600 milliseconds to 250 milliseconds. Subjects were instructed to verbalize the presented number in the same manner as the treatment group. As subjects improved, the speed of number presentation was regularly increased.

At the conclusion of the 6 week training, subjects in both treatment and placebo groups were re-tested with the WIAT and K-D tests by trained test administrators masked to the sequence of intervention. All testing procedures were identical to pre-treatment testing Control group subjects were subsequently crossed over to complete the same 6 week treatment protocol of the treatment group then tested post-treatment.

To evaluate the long-term effects, a convenience sample of subjects still enrolled at the school at a 1 year follow-up (n=25), were evaluated with identical testing procedures used during the pre-treatment testing.

Statistical Analysis

Statistical analyses were performed using Stata 12.0 software (StataCorp, College Station, Tex.). Differences in reading fluency percentile rank from pre-treatment and post-treatment were calculated and compared using two sample t-tests. Differences in the K-D time score from pre-treatment and post-treatment were calculated for kindergarteners and compared using two sample t-tests. Statistical significance was set at p<0.05.

Results

Characteristics of the study cohort are shown in Table 1. Age, gender, ethnicity and grade distributions were similar between the treatment and control groups. Reading fluency score data is shown in Table 2. Subjects in the treatment group had significantly higher reading fluency scores after treatment (p<0.001, FIG. 1) and post-treatment scores were significantly higher compared to the control group (p<0.005, FIG. 1). At the 1 year follow-up, reading fluency scores were significantly higher than post-treatment scores for subjects in grade 1 (p=0.037). Additionally, these 1 year follow-up scores were higher than pre-treatment scores across all grades, with an average improvement of 17 percentile rank points across all grades in the treatment group.

Kindergarten K-D composite score data is shown in FIG. 3. The treatment group had a significant improvement (lower score) in their post-treatment K-D composite score compared to pre-treatment (p<0.001) and compared to the control group (p=0.0435). Subjects in the treatment group also had a greater percentage change (p<0.001) in their K-D composite scores compared to the control group. Additionally at the 1 year follow-up, K-D composite scores demonstrated improvement compared to post-treatment scores (p<0.005). The 1st grade level reading fluency scores of these students at the 1 year follow-up was 5 percentile points higher than the naïve 1st grade students' pre-treatment reading fluency scores (43rd vs. 38th percentile rank).

Discussion

The results of this study demonstrate that the K-D remediation training has the ability to improve reading fluency in a 6 hour program across 6 weeks of training. We hypothesize that this improvement in reading fluency is a result of rigorous practice of eye movements, both saccades and vergence, and shifting visuo-spatial attention, all of which are vital to the act of reading.

The treatment group subjects in grades 1 through 3 had higher post-treatment standardized reading fluency scores compared to the control group demonstrating that the training protocol improved reading fluency. Subjects in the control group did not significantly improve in their standardized reading scores after the 6 weeks. Additionally, kindergarteners had improvement in their K-D composite scores compared to controls. Furthermore, the 1 year follow-up results indicate that the treatment effect persisted with no significant worsening of reading fluency scores across all grades and in fact students had on average higher 1 year follow-up reading fluency scores when compared to students of the same grade before treatment. Subjects who received training during kindergarten had higher reading fluency scores in 1st grade at the 1 year follow-up compared to 1st grade students who had not yet received training. This demonstrates that early intervention with K-D Remediation may help to improve reading fluency, which may be explained by a critical learning period during brain development and correlates with previous study findings of greater improvements in reading fluency observed in Grade 2 and 3 compared to Grade 4 following a 6 week training regimen.

Limitations of this study include small sample size, limited subject demographics, lack of double blinding, and absence of 1 year follow-up on control subjects due to the crossover design. Future research should expand across a larger cohort, include a variety of demographics across multiple schools, use a double blind study design and study the long-term performance of control subjects for comparison. Also, history of learning disability and other clinical conditions that may affect reading performance should be evaluated concurrently to determine if there are correlations with amount of improvement. Additionally the training course utilized in this study was 6 weeks in length. Further study should determine an optimal training length to maximize reading fluency outcomes.

Improving reading fluency is critical to American youth as the National Assessment of Educational Progress recently reported that children who aren't reading proficiently by grade 4 are four times likely to drop out of high school. Additionally, it is estimated that only 75% of America's high school students graduate leaving behind approximately 1.3 million students annually that are failing to complete high school. These staggering dropout rates translate to significant social and economic costs to the rest of the nation. Those who do not have a high school diploma will earn over the course of their lifetime less than a high school graduate. Also it is estimated that dropouts from the class of 2010 alone will cost the nation more than $337 billion in lost wages over the course of their lifetimes.

Conclusions

In this pilot study, the K-D remediation software has shown to improve reading fluency in a 6-week therapy program conducted in a school setting without requiring any additional use of specialized teachers. Eye movements only represent one facet of reading comprehension. There are many other factors that are involved such as cognitive processing, attention span and intellectual ability which may play different roles in an individual student's reading success. Nonetheless, the results of this pilot study suggest that the K-D remediation software may be effective in significantly improving reading fluency through rigorous practice of eye movements, visuo-spatial attention, and oculomotor integration in a 6 week therapy program. The K-D methodology merits further testing as a tool to improve reading fluency of our youth in a larger clinical trial.

### Exemplary Embodiments

A method can use a computer having a visual output device, the computer being configured to visually display numbers in spatial and temporal arrangements on the visual output device, and the method can include (a) visually presenting the visual output device to a trainee, (b) instructing the trainee to read aloud numbers displayed on the visual output device in a specified order without moving the trainee's head, (c) visually displaying to the trainee a predetermined spatial and temporal arrangement of numbers on the visual output device, (d) reading aloud the numbers in the visually displayed predetermined spatial and temporal arrangement, wherein reading is to be carried out by the trainee without moving the trainee's head, thereby causing the trainee to engage in saccadic eye movement while reading the numbers, and (e) repeating steps (c) and (d) on a predetermined schedule.

In some such methods, the predetermined spatial and temporal arrangement of numbers includes numbers spaced apart along a sequence of vertically spaced apart horizontal axes. In some such methods, a different predetermined spatial and temporal arrangement is visually displayed each time step (c) is repeated. In some such methods, the predetermined spatial and temporal arrangement of numbers is at least partially randomized each time steps (c) and (d) are repeated.

Some such methods also include (f) measuring the time the trainee takes to read aloud the numbers in the visually displayed predetermined spatial and temporal arrangement. Some such methods also include repeating step (f) along with steps (c) and (d) on the predetermined schedule. Some such methods also include (g) if a measured-time adjustment parameter has been previously determined, determining the predetermined spatial and temporal arrangement of numbers based on the measured-time adjustment parameter before step (a), and (h) based on the time measured in step (f), determining a measured-time adjustment parameter. In such methods step (e) can further include repeating steps (g) and (h) along with steps (c), (d) and (f) on the predetermined schedule. In some such methods, adjusting the predetermined spatial and temporal arrangement of numbers based on the measured-time adjustment parameter can include adjusting a time during which at least a number in the predetermined spatial and temporal arrangement of numbers is displayed and/or the relative locations of at least two numbers in the predetermined spatial and temporal arrangement of numbers. In some such methods step (f) is carried out by the computer, and the computer can automatically record the measured time. In some such methods step (f) is carried out by a trainer who can be someone other than the trainee or can be the trainee himself. In some such methods the trainer can input to the computer the measured time by way of an input device included in the computer.

Some such methods include (i) counting the number of errors the trainee makes reading aloud the numbers in the visually displayed predetermined spatial and temporal arrangement. In some such methods, step (e) also includes repeating step (i) along with steps (c) and (d) on the predetermined schedule. Some such methods include (j) if a counted-errors adjustment parameter has been previously calculated, determining the predetermined spatial and temporal arrangement of numbers based on the counted-errors adjustment parameter before step (a), and (k) based on the number of errors counted in step (i), determining a counted-errors adjustment parameter. In some such methods step (e) also includes repeating steps (j) and (k) along with steps (c), (d) and (i) on the predetermined schedule. In some such methods adjusting the predetermined spatial and temporal arrangement of numbers based on the counted-errors adjustment parameter includes adjusting a time during which at least a number in the predetermined spatial and temporal arrangement of numbers is displayed. In some such methods adjusting the predetermined spatial and temporal arrangement of numbers based on the counted-errors adjustment parameter includes adjusting the relative locations of at least two numbers in the predetermined spatial and temporal arrangement of numbers. In some such methods step (i) is carried out by the computer. In some such methods step (i) is carried out by a trainer who can be someone other than the trainee or can be the trainee himself. In some such methods the trainer can input to the computer the counted number of errors by way of an input device included in the computer.

In some such methods the predetermined spatial and temporal arrangement of numbers includes each number in the arrangement being displayed only for a predetermined time. In some such methods the predetermined time is less than 3 seconds, less than 2 seconds, between 0.25 and 1.6 seconds, about 1 second or about 0.5 seconds. In some such methods each number in the predetermined spatial and temporal arrangement of numbers is displayed while no other number is displayed. In some such methods all numbers in the predetermined spatial and temporal arrangement of numbers are simultaneously displayed.

In some such methods the predetermined schedule includes at least one 20-minute training period. In some such methods the training period is repeated no more than once a day, and/or three days a week, and/or for a total of six weeks. In some such methods repetition on the predetermined schedule results in an improved reading fluency on the part of the trainee. In some such methods the predetermined schedule includes stopping the training once a predetermined improvement in reading fluency on the part of the trainee is achieved.

In some such methods the computer is, for example, a smart phone, a tablet computer, a e-reader, a notebook computer, a laptop computer, and/or a desktop computer. In some such methods the computer further includes a connection, the connection being operably connected to a memory which can be, for example locally contained within the computer or remotely located on a server connected to the computer by way of a network. Some such methods further include (l) transmitting, from the computer, through the connection, to the memory, a signal representative of the fact that the trainee has attempted to carry out the method.

In some such methods, the trainee has been previously selected to participate in the method because the trainee has been previously diagnosed with dyslexia. In some such methods, the trainee has been previously selected to participate in the method because the trainee has been previously diagnosed with traumatic brain injury.

Methods of rehearsing saccadic eye movement can use a visual display device including a plurality of non-transient predetermined spatial arrangements of numbers. Such methods can include (a) visually presenting to a trainee the visual display device and a chosen predetermined spatial arrangement of the plurality of predetermined spatial arrangements of numbers, (b) instructing the trainee to read aloud the numbers in the chosen predetermined spatial arrangement in a specified order without moving the trainee's head, (c) visually displaying to the trainee the chosen predetermined spatial arrangement of numbers on the visual display, (d) reading aloud the numbers in the chosen predetermined spatial arrangement, wherein reading is to be carried out by the trainee without moving the trainee's head, thereby causing the trainee to engage in saccadic eye movement while reading the numbers, and (e) repeating steps (c) and (d) on a predetermined schedule.

In some such methods, the plurality of non-transient predetermined spatial arrangements of numbers is printed. In some such methods, the plurality of non-transient predetermined spatial arrangements of numbers is printed on the page of a bound test booklet. In some such methods, each of the plurality of non-transient predetermined spatial arrangements of numbers is printed on a separate card, the plurality of separate cards being unbound.

Methods of improving reading fluency can include identifying a trainee in need of improved reading fluency, the trainee undertaking a predetermined series of saccadic eye movements, the trainee repeating the predetermined series of saccadic eye movements on a predetermined schedule, thereby improving the trainee's reading fluency. In some such methods, the trainee has been previously selected to participate in the method because the trainee has been previously diagnosed with dyslexia and/or traumatic brain injury.

The various features and elements of the exemplary embodiments are generally combinable with one another in a variety of permutations, all of which are within the scope of this disclosure.

I claim:

1. A method of using a computer, the computer comprising a visual output device, the computer being configured to visually display numbers in spatial and temporal arrangements on the visual output device, the method comprising:
(a) visually presenting the visual output device to a trainee;
(b) instructing the trainee to read aloud numbers displayed on the visual output device in a specified order without moving the trainee's head;
(c) visually displaying to the trainee a predetermined spatial and temporal arrangement of numbers on the visual output device;
(d) reading aloud the numbers in the visually displayed predetermined spatial and temporal arrangement, wherein reading is to be carried out by the trainee without moving the trainee's head, thereby causing the trainee to engage in saccadic eye movement while reading the numbers; and
(e) repeating steps (c) and (d) on a predetermined schedule.

2. The method of claim 1 wherein the predetermined spatial and temporal arrangement of numbers includes numbers spaced apart along a sequence of vertically spaced apart horizontal axes.

3. The method of claim 1 further comprising:
(f) measuring the time the trainee takes to read aloud the numbers in the visually displayed predetermined spatial and temporal arrangement.

4. The method of claim 3 wherein step (e) further comprises repeating step (f) along with steps (c) and (d) on the predetermined schedule.

5. The method of claim 4 further comprising:
(g) if a measured-time adjustment parameter has been previously determined, determining the predetermined spatial and temporal arrangement of numbers based on the measured-time adjustment parameter before step (a); and
(h) based on the time measured in step (f), determining a measured-time adjustment parameter;
wherein step (e) further comprises repeating steps (g) and (h) along with steps (c), (d) and (f) on the predetermined schedule.

6. The method of claim 5 wherein adjusting the predetermined spatial and temporal arrangement of numbers based on the measured-time adjustment parameter includes adjusting a time during which at least a number in the predetermined spatial and temporal arrangement of numbers is displayed.

7. The method of claim 5 wherein adjusting the predetermined spatial and temporal arrangement of numbers based on the measured-time adjustment parameter includes adjusting the relative locations of at least two numbers in the predetermined spatial and temporal arrangement of numbers.

8. The method of claim 6 wherein adjusting the predetermined spatial and temporal arrangement of numbers based on the measured-time adjustment parameter includes adjusting the relative locations of at least two numbers in the predetermined spatial and temporal arrangement of numbers.

9. The method of claim 3 wherein step (f) is carried out by the computer.

10. The method of claim 9 wherein the computer automatically records the measured time.

11. The method of claim 1 wherein the predetermined spatial and temporal arrangement of numbers includes each number in the arrangement being displayed only for a predetermined time.

12. The method of claim 1 wherein each number in the predetermined spatial and temporal arrangement of numbers is displayed while no other number is displayed.

13. The method of claim 1 wherein all numbers in the predetermined spatial and temporal arrangement of numbers are simultaneously displayed.

14. The method of claim 1 wherein the predetermined schedule includes at least one 20-minute training period.

15. The method of claim 14 wherein the training period is repeated no more than once a day.

16. The method of claim 15 wherein the training period is repeated three days a week.

17. The method of claim 16 wherein the training period is repeated for a total of six weeks.

18. The method of claim 1 wherein repetition on the predetermined schedule results in an improved reading fluency on the part of the trainee.

19. The method of claim 1 wherein the computer is a smart phone, a tablet computer, a notebook computer, or a desktop computer.

20. The method of claim 1 wherein the computer further comprises a connection, the connection being operably connected to a memory, the method further comprising:
(l) transmitting, from the computer, through the connection, to the memory, a signal representative of the fact that the trainee has attempted to carry out the method;
wherein the memory is either:
locally contained within the computer; or
remotely located on a server connected to the computer by way of a network.

21. The method of claim 1 wherein the trainee has been previously selected to participate in the method because the trainee has been previously diagnosed with dyslexia.

22. The method of claim 1 wherein the trainee has been previously selected to participate in the method because the trainee has been previously diagnosed with traumatic brain injury.

23. A method of rehearsing saccadic eye movement using a visual display device including a plurality of non-transient predetermined spatial arrangements of numbers, the method comprising:
(a) visually presenting to a trainee the visual display device and a chosen predetermined spatial arrangement of the plurality of predetermined spatial arrangements of numbers;
(b) instructing the trainee to read aloud the numbers in the chosen predetermined spatial arrangement in a specified order without moving the trainee's head;
(c) visually displaying to the trainee the chosen predetermined spatial arrangement of numbers on the visual display;
(d) reading aloud the numbers in the chosen predetermined spatial arrangement, wherein reading is to be carried out by the trainee without moving the trainee's head, thereby causing the trainee to engage in saccadic eye movement while reading the numbers; and
(e) repeating steps (c) and (d) on a predetermined schedule.

24. A method of improving reading fluency, the method comprising:
identifying a trainee in need of improved reading fluency;
the trainee undertaking a predetermined series of saccadic eye movements;
the trainee repeating the predetermined series of saccadic eye movements on a predetermined schedule, thereby improving the trainee's reading fluency.
